SEQUENCE LISTING (12 sequences)

SEQ ID NO: 1 - DNA, 20 bp, Artificial Sequence
  Primer
cgaatgactggattgtggct

SEQ ID NO: 2 - DNA, 20 bp, Artificial Sequence
  Primer
tgagttctgtcgggacacag

SEQ ID NO: 3 - DNA, 18 bp, Artificial Sequence
  Primer
cttccgcgacgtggacat

SEQ ID NO: 4 - DNA, 18 bp, Artificial Sequence
  Primer
gttgggcggcaggtactg

SEQ ID NO: 5 - DNA, 21 bp, Artificial Sequence
  Primer
caacactgccaacgtccagat

SEQ ID NO: 6 - DNA, 24 bp, Artificial Sequence
  Primer
tcttgcagtggtaggtgatgttct

SEQ ID NO: 7 - DNA, 20 bp, Artificial Sequence
  Primer
acacgtggggacattagtgg

SEQ ID NO: 8 - DNA, 20 bp, Artificial Sequence
  Primer
gtggaatgcagaggtggttt

SEQ ID NO: 9 - DNA, 23 bp, Artificial Sequence
  Primer
tctcctctgcattcaacagcgac

SEQ ID NO: 10 - DNA, 21 bp, Artificial Sequence
  Primer
ccctgttgctgtagccaaatt

SEQ ID NO: 11 - DNA, 20 bp, Artificial Sequence
  Primer
gtgcagagtccagcaaaggt

SEQ ID NO: 12 - DNA, 19 bp, Artificial Sequence
  Primer
cgataggcctcctgaaagc

(19) United States
(12) Patent Application Publication
(10) Pub. No.: US 2018/0169136 A1
(43) Pub. Date: Jun. 21, 2018

(54) USE OF PHARMACEUTICAL COMPOSITION IN PREPARATION OF DRUG FOR PROMOTING CHONDROCYTE GENERATION

(71) Applicant: KAOHSIUNG MEDICAL UNIVERSITY, Kaohsiung City (TW)

(72) Inventors: Mei-ling Ho, Kaohsiung City (TW); Je-Ken Chang, Kaohsiung City (TW); Shun-Cheng Wu, Kaohsiung City (TW); Chih-Hsiang Chang, Kaohsiung City (TW)

(73) Assignee: KAOHSIUNG MEDICAL UNIVERSITY, Kaohsiung City (TW)

(21) Appl. No.: 15/737,312

(22) PCT Filed: Jun. 18, 2015
(86) PCT No.: PCT/CN2015/081835

§ 371 Date: Dec. 17, 2017

(51) Int. Cl. A61K 31/728; A61K 38/39; A61K 35/28; A61K 31/366; A61K 9/16; A61P 19/02

(52) U.S. Cl. CPC A61K 31/728; A61K 38/39; A61K 35/28; A61K 2300/00; A61K 9/16; A61P 19/02; A61K 31/366

(57) ABSTRACT

The present invention relates to a use of a pharmaceutical composition in the preparation of a drug for promoting chondrogenesis, wherein the pharmaceutical composition comprises a hyaluronic acid mixture and a statin compound.

This application contains a Sequence Listing in computer readable form. The computer readable form is incorporated herein by reference. This application is the National Stage of International Application No. PCT/CN2015/081835 filed on Jun. 18, 2015, which is incorporated herein by reference.

### FIELD OF THE INVENTION

The present invention relates to a use of a pharmaceutical composition in preparation of a drug for promoting chondrogenesis, specifically, a pharmaceutical composition comprising hyaluronic acid and simvastatin for promoting chondrogenesis.

### BACKGROUND OF THE INVENTION

Cartilage tissue is a connective tissue lacking blood vessels, lymphatic system and nerves, composed primarily of hyaline cartilage, and hyaline cartilage is composed primarily of chondrocytes, type II collagen, and proteoglycan. Once the cartilage tissue is damaged, the number of chondrocytes located in the immediate vicinity is too limited to repair the damage, moreover, it is too difficult for chondrocytes to migrate to the damaged site because they are coated by extracellular matrix. It is currently known that most of the new tissues generated by cartilage self-repair is fibrocartilage tissue which is primarily type I collagen. Fibrocartilage will gradually degrade because it lacks the biomechanics of cartilage and the function of hyaline cartilage, which makes it difficult for joints to regain normal pre-injury conditions for movement. Applications of tissue engineering to cartilage tissue repair have been rapidly developing in recent years. In this approach, chondrocytes or mesenchymal stem cells are used to generate viable and functional articular cartilage tissues. The use of mesenchymal stem cells as a cell source is currently acknowledged as an approach of high potential. Although mesenchymal stem cells are capable of being differentiated into chondrocytes, if they are to be used to repair articular cartilage tissues, inducible factors are needed to induce chondrogenic differentiation in mesenchymal stem cells. However, inducible factors of protein origins are expensive and involve with problems such as protein denaturation or pathogen contamination. When chemical inducible factors are used, other side effects are involved. It has been reported in the past that bone morphogenetic protein-2 (BMP-2) is capable of inducing chondrogenesis in stem cells and inducing ossification in osteoblasts, therefore can be used in articular cartilage and bone tissue engineering. However, there is a concern about the risk of osteosclerosis when articular cartilage is repaired by inducing the expression of BMP-2. Previous studies have already reported that though cartilage defects can be repaired by inducing high expression of BMP-2 in stem cells, bone spurs may be formed at the repaired sites (Arthritis Rheum. Articular cartilage repair by gene therapy using growth factor-producing mesenchymal cells. 2003 February; 48 (2): 430-41.) How to stimulate stem cells to express large amounts of BMP-2 while avoiding ossification caused by BMP-2 is still an unsolved problem.

Statin drugs, including simvastatin, are mainly used clinically to lower blood lipids. Moreover, they can induce expression of BMP-2 in chondrocytes, production of chondrogenic genes (type II collagen and aggrecan) and extracellular matrix. Statin drugs also can induce expression of BMP-2 in osteoblasts to repair bone fractures by enhancing ossification. Therefore, though statin drugs can be used to induce BMP-2 expression for cartilage repair, how to induce high expression of BMP-2 in stem cells to repair cartilage defects while avoiding ossification caused by BMP-2 is yet an unsolved problem.

### SUMMARY OF THE INVENTION

The present invention relates to a use of a pharmaceutical composition in the preparation of a drug for promoting chondrogenesis, wherein the pharmaceutical composition comprises a hyaluronic acid mixture and a statin compound.

### BRIEF DESCRIPTION OF THE DRAWINGS

FIG. 1 shows the effect of simvastatin on the expression of sulfated glycosaminoglycans (sGAG) in human adipose-derived stem cells (hADSCs) cultured in wells coated with hyaluronic acid (HA) or without hyaluronic acid (* and **: as compared to the group not treated with hyaluronic acid and simvastatin, *p<0.05; **p<0.01. #: as compared to the group treated with simvastatin (0 μM), P<0.01. The number of experiments is 3).

FIG. 2A shows the effect of simvastatin on the expression of chondrogenic gene Sox-9 in human adipose-derived stem cells cultured with hyaluronic acid (HA) or without hyaluronic acid (non-HA) (as compared to the group with no hyaluronic acid (non-HA), **p<0.01. The number of experiments is 4).

FIG. 2B shows the effect of simvastatin on the expression of chondrogenic gene aggrecan in human adipose-derived stem cells cultured with hyaluronic acid (HA) or without hyaluronic acid (Non-HA) (as compared to the group with no hyaluronic acid (non-HA), **p<0.01. The number of experiments is 4).

FIG. 2C shows the effect of simvastatin on the expression of chondrogenic gene Col II in human adipose-derived stem cells cultured with hyaluronic acid (HA) or without hyaluronic acid (Non-HA) (as compared to the group with no hyaluronic acid (non-HA), **p<0.01. The number of experiments is 4).

FIG. 3A shows the effect of simvastatin on gene expression of BMP-2 in human adipose-derived stem cells cultured with hyaluronic acid (HA) or without hyaluronic acid (Non-HA) (as compared to the data of the first day of the group with no hyaluronic acid (non-HA), **p<0.01. The number of experiments is 4).

FIG. 3B shows the effect of simvastatin on gene expression of osteocalcin in human adipose-derived stem cells cultured with hyaluronic acid (HA) or without hyaluronic acid (Non-HA) (as compared to the data of the first day of the group with no hyaluronic acid (non-HA), **p<0.01. The number of experiments is 4).

FIG. 4 is the result analysis of the formation and expression of sulfated glycosaminoglycans (sGAG) in human adipose-derived stem cells coated with a three-dimensional hyaluronic acid/fibrin hydrogel and treated with simvastatin (as compared to the hyaluronic acid group (HA) and hyaluronic acid plus Simvastatin group (HA+Sim) on the same days, **p<0.01. The number of experiments is 3).

FIG. 5 is the result analysis of the expression of type II collagen (Col II) in human adipose-derived stem cells coated with a three-dimensional hyaluronic acid/fibrin hydrogel and treated with simvastatin (as compared to the hyaluronic acid (HA) group and hyaluronic acid plus Simvastatin (HA+Sim) group on the same days, **p<0.01. The number of experiments is 3).

FIG. 6A is the result analysis of the expression of chondrogenic gene Sox-9 in human adipose-derived stem cells coated with a three-dimensional hyaluronic acid/fibrin hydrogel and treated with simvastatin.

FIG. 6B is the result analysis of the expression of chondrogenic gene aggrecan in human adipose-derived stem cells coated with a three-dimensional hyaluronic acid/fibrin hydrogel and treated with simvastatin.

FIG. 6C is the result analysis of the expression of chondrogenic gene Col II by human adipose-derived stem cells coated with a three-dimensional hyaluronic acid/fibrin hydrogel and treated with simvastatin.

FIG. 7 shows the results of the articular cartilage defects in the knee joints of miniature pigs treated with the three-dimensional hyaluronic acid/fibrin hydrogel containing simvastatin and human adipose-derived stem cells (arrows show where the knee joint are repaired).

### DETAILED DESCRIPTION OF THE INVENTION

The present invention has demonstrated by experiments that when adipose-derived stem cells are treated with a hyaluronic acid-containing microenvironment and simvastatin, not only the production of BMP-2, chondrogenic genes (Sox-9, aggrecan and collagen Type II), and articular cartilage extracellular matrix (sGAG) can be enhanced, the expression of osteocalcin induced by simvastatin can be reduced at the same time to suppress osteosclerosis, which can be used to repair cartilage defects.

The present invention discovers that simvastatin together with hyaluronic acid have the effect of inducing chondrogenesis in stem cells. In addition, the expression of BMP-2 is more effectively enhanced, the expression of chondrogenic genes is increased, and the level of sulfated glycosaminoglycan (a major extracellular matrix in articular cartilage) produced by adipose-derived stem cells is increased when a three-dimensional hydrogel carrier is further formed from fibrin to coat hyaluronic acid and stem cells, and a simvastatin-containing microsphere is also added into the carrier. In addition, it is found that the present composition (combining adipose-derived stem cells with simvastatin and hyaluronic acid) effectively repairs articular cartilage defects in a miniature pig cartilage defect model.

As used herein, the term “a” or “an” are employed to describe elements and components of the present invention. This is done merely for convenience and to give a general sense of the present invention. This description should be read to include one or at least one and the singular also includes the plural unless it is obvious that it is meant otherwise.

The present invention provides a pharmaceutical composition comprising a hyaluronic acid-containing mixture and a statin drug, a dose kit, and a therapeutic method.

The present invention provides a use of a pharmaceutical composition in the preparation of a drug for promoting chondrogenesis, wherein the pharmaceutical composition comprises a hyaluronan-containing mixture and a statin compound.

As used herein, the term “promoting chondrogenesis” includes, but is not limited to, promoting cells to synthesize, metabolize, express or secrete factors or hormones associated with chondrogenic differentiation so that the cells form chondrocytes.

In one embodiment, the hyaluronic acid-containing mixture comprises hyaluronic acid, fibrin and a stem cell. In a preferred embodiment, the fibrin forms a hydrogel. The fibrin form a scaffold structure of a three-dimensional hydrogel, i.e., a 3D fibrin hydrogel, which is used as a carrier to coat the hyaluronic acid and the stem cell.

As used herein, the term “hydrogel” refers to a gel using water as a dispersion medium, constituted by introducing a partially hydrophobic group and hydrophilic residues into a water-soluble polymer having a network-like cross-linked structure to form a cross-linked polymer, wherein water molecules are connected within the network, and the hydrophobic residues are water-swollen. Therefore, any water-soluble or hydrophilic polymers can from hydrogels through certain chemical or physical crosslink. Hydrogels can be categorized into physical hydrogels and chemical hydrogels: (1) physical hydrogels are formed by physical forces such as electrostatic interactions, hydrogen bonding, chain entanglement, etc. Such gels are non-permanent, can be transformed into solution under heat, they are also known as pseudogels or thermoreversible gels. Many natural polymers are in a state of stable hydrogels at room temperature, such as kappa-2 carrageenan, agar; as for synthetic polymers, polyvinyl alcohol (PVA) is a typical example, a hydrogel which is stable below 60° C. and can be obtained after a freezing and thawing treatment. (2) Chemical hydrogels are three-dimensional network polymers crosslinked by chemical bonds, they are permanent and also known as true gels. There are macroscopic hydrogels and microscopic hydrogels (microspheres) which are categorized according to the shape and the size of each hydrogel. According to the shape, macroscopic hydrogels can be further categorized into columnar, porous sponge-like, fibrous, film-like, spherical hydrogels. Currently, prepared microspheres are categorized into micron-sized hydrogels and nano-sized hydrogels.

As used herein, the term “stem cell” refers to a group of cells that are capable of self-renewal and proliferation while maintaining an undifferentiated state over a long period of time. In addition, this group of cells have multi-differentiation potential, capable of differentiating into cell populations of different lineages and functionally specific tissues after being properly induced and stimulated. The term “human mesenchymal stem cells” refers to any cells which are derived from human stroma tissue and capable of unlimited self-renewal and differentiating into a variety of cells or tissue types, such as, but not limited to, one selected from the group consisting of adipose-derived mesenchymal stem cells, bone marrow-derived mesenchymal stem cells, umbilical cord-derived mesenchymal stem cells, periosteum-derived mesenchymal stem cells, synovium-derived mesenchymal stem cells, and muscle-derived mesenchymal stem cells. The examples of the present invention are illustrated by the examples of human adipose-derived mesenchymal stem cells (or known as human adipose-derived stem cells, hADSCs). However, the present invention is not limited by the following examples. In one embodiment, the stem cells comprise a human dermal fibroblast, a mesenchymal stem cell or an adipose-derived stem cell. In a preferred embodiment, the stem cells comprise human adipose-derived stem cells (hADSCs).

In one embodiment, the concentration of the hyaluronic acid ranges from 0.1 wt % to 10 wt %. In a preferred embodiment, the concentration of the hyaluronic acid ranges from 0.5 wt % to 5 wt %. In a more preferred embodiment, the concentration of the hyaluronic acid is 1 wt %.

As used herein, the term “statin compound” includes, but is not limited to, atorvastatin, cerivastatin, fluvastatin, lovastatin, mevastatin, pitavastatin, pravastatin, rosuvastatin or simvastatin. In a preferred embodiment, the statin compound is simvastatin. The structural of simvastatin is as following formula (I):

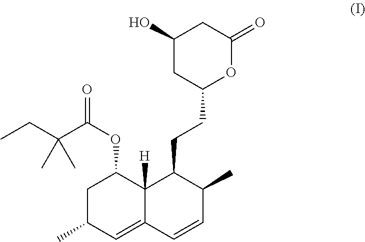 [SMILES: CCC(C)(C)C(=O)O[C@H]1C[C@@H](C)C=C2C=C[C@H](C)[C@H](CC[C@@H]3C[C@@H](O)CC(=O)O3)[C@H]21]

In one embodiment, the concentration of simvastatin ranges from 0.1 μM to 10 μM. In a preferred embodiment, the concentration of simvastatin ranges from 0.5 μM to 5 μM. In a more preferred embodiment, the concentration of simvastatin is 1 μM. In another embodiment, the simvastatin is in the dosage form of a microsphere. The microsphere dosage form can effectively control the concentration of the released simvastatin.

Thus, the pharmaceutical composition of the present invention can be administered to a subject as a composition comprising a fibrillar hydrogel having hyaluronic acid and stem cells, and a simvastatin-containing microsphere to promote chondrogenesis or to repair cartilage defects. In addition, the simvastatin-containing microsphere can be administered to the subject by being directly added into the fibrillar hydrogel having hyaluronic acid and stem cells.

As used herein, the term “cartilage defects” includes, but is not limited to, articular cartilage degeneration or diseases of articular cartilage defects/wear caused by gene mutations or damages cause by external force. The articular cartilage widely exists in the articular surface of a bone, costal cartilage, trachea, pinna, lumbar discs.

It is currently known that many factors or triggering agents stimulate anabolic metabolism of chondrocytes, for example, insulin-like growth factor-I (IGF-I) is the principal growth factor for anabolic metabolism in synovial fluid, it also stimulates the synthesis of proteoglycans and collagens; in addition, members of the bone morphogenic protein (BMP) family (in particular BMP2, BMP4, BMP6 and BMP7) and members of the human transforming growth factor-β (TGF-β) family can induce the anabolic metabolism of chondrocytes (Chubinskaya and Kuettner, 2003). Further, a compound which can induce the anabolic metabolism of chondrocytes has been identified in recent years (U.S. Pat. No. 6,500,854; EP 1 391 211). Therefore, in a preferred embodiment, the effect of promoting chondrogenesis of the present invention is to induce chondrification in cells or stem cells by increasing or enhancing and stimulating factors or hormones associated with the anabolic metabolism of chondrocytes. In a more preferred embodiment, the promoting chondrogenesis is achieved by increasing the expression of bone morphogenetic protein-2 (BMP-2).

Further, two major components of the extracellular matrix of the articular cartilage are proteoglycan and type II collagen (Col II). Glycosaminoglycan (GAG) is the glycan moiety in proteoglycan, the structure of which is characterized by having sulfated disaccharide repeating units. Type II collagen and proteoglycans form the framework of cartilage, providing the cartilage with stability and elasticity. In addition to being the structural framework of tissues, the extracellular matrix (ECM) modulates chondrocytes through cellular signaling mechanisms. These complex interactions affect gene expression in chondrocytes at the mRNA and protein expression level, altering the intracellular state of the cells. In addition, the degradation of extracellular matrix is often associated with the pathological state of the cartilage, in which the expression level of type II collagen is reduced and the viability of matrix metalloproteinases (MMPs) is enhanced. Further, growth factors such as TGF-β1 and IGF-I are involved in the secretion and maintenance of the extracellular matrix of articular chondrocytes. Therefore, in a preferred embodiment, the efficacy of the present invention for promoting chondrogenesis is achieved by increasing the production of extracellular matrix and stimulating the expression of factors associated with the maintenance/synthesis of the extracellular matrix, thereby inducing chondrogenesis of cells or stem cells to form cartilage. In a more preferred embodiment, the promotion of chondrogenesis is achieved by increasing the production of sulfated glycosaminoglycans (sGAG).

Chondrogenic genes can induce chondrogenesis of cells, chondrogenic genes include, but are not limited to, Sox-9, aggrecan or type II collagen. In one embodiment, the promotion of chondrogenesis is achieved by enhancing the expression of chondrogenic genes, wherein the chondrogenic genes comprise Sox-9, aggrecan or type II collagen.

As used herein, the term “expression” includes, but is not limited to, the expression of genes, RNAs, or proteins.

In addition, most compounds that stimulate the anabolic metabolism of chondrocytes have serious side effects, for example, overexpression of BMP-2 leading to the formation of bone spurs, i.e., occurrence of osteosclerosis. Therefore, currently there is no compound that can stimulate chondrogenic differentiation without such side effects. Osteogenic factors or factors inducing osteosclerosis include, but are not limited to, osteocalcin. Therefore, the present invention not only promotes chondrogenesis by strengthening various factors associated with chondrogenesis, but also avoids the occurrence of osteosclerosis while chondrocytes are produced. In one embodiment, the pharmaceutical composition further inhibits osteosclerosis of the produced chondrocytes. In a preferred embodiment, the inhibition of osteosclerosis of the produced chondrocytes is achieved by inhibiting the expression of osteocalcin.

The present invention also provides a method for treating cartilage defects comprising administering a therapeutically effective amount of pharmaceutical composition to a cartilage defect site of a subject, wherein the pharmaceutical composition comprises a hyaluronic acid (HA) mixture and a statin compound. In one embodiment, the hyaluronic acid mixture comprises hyaluronic acid, fibrin, and a stem cell. In a preferred embodiment, the fibrin forms a hydrogel. The fibrin forms the scaffold structure of a three-dimensional hydrogel, that is a 3D fibrin hydrogen, which is used as a carrier for coating the hyaluronic acid and the stem cell.

In another embodiment, the statin compound is simvastatin. The statin compound of the present invention, such as simvastatin, may be embedded in, for example, microcapsules prepared by coacervation techniques or by interfacial polymerization (e.g. carboxymethyl cellulose or gelatin microcapsules, respectively, and poly-(methyl methacrylate) microcapsules), colloidal drug delivery systems (for example, liposomes, microspheres, microemulsions, nanoparticles or nanocapsules), or macroemulsions. In a preferred embodiment, the simvastatin is in a dosage form of a microsphere. The microsphere can effectively control the concentration of the released simvastatin.

As used herein, the term “therapeutically effective amount” refers to an amount of a compound, when administered to a subject to treat a disease, that is sufficient to effect the desired treatment of the disease. The “therapeutically effective amount” may vary depending on the compound, the disease and its severity, and the age, weight, etc. of the subject to be treated. In one embodiment, the therapeutically effective amount of the hyaluronic acid ranges from 0.1 to 10 wt %. In a preferred embodiment, the therapeutically effective amount of the hyaluronic acid ranges from 0.5 to 5 wt %. In a more preferred embodiment, the therapeutically effective amount of the hyaluronic acid is 1 wt %. In another embodiment, the therapeutically effective amount of the simvastatin ranges from 0.1 to 10 μM. In a preferred embodiment, the therapeutically effective amount of the simvastatin ranges from 0.5 to 5 μM. In a more preferred embodiment, the therapeutically effective amount of the simvastatin is 1 μM.

The term “treat” refers to any improvements of a disease or illness (also refers to inhibition of the disease or amelioration of the appearance, extent or severity of at least one of its clinical symptoms).

As used herein, the term “subject” refers to an animal. In a preferred embodiment, the subject refers to a mammal. In a more preferred embodiment, the subject refers to a human.

The pharmaceutical compositions of the present invention may be prepared in a manner well known in the art of pharmacy. In general, a pharmaceutically effective amount of the pharmaceutical composition of the present invention is administered. The dose of the pharmaceutical composition actually administered is generally determined by physicians in accordance with the following relevant conditions, including: the illness to be treated; the selected route of administration; the compound actually administered; the age, weight and response of each individual patient; the severity of the patient's symptoms; and the likes.

The preferred route of administration of the pharmaceutical composition of the present invention to the subject is intraarticular administration. The pharmaceutical compositions of the present invention may also be administered in a sustained release dosage form or administered through a sustained release drug delivery system.

The present invention further provides a kit, which comprises a hyaluronic acid mixture and a statin compound.

In one embodiment, the hyaluronic acid mixture comprises hyaluronic acid (HA), fibrin, and a stem cell. In a preferred embodiment, the fibrin forms a hydrogel. The fibrin forms the scaffold structure of a three-dimensional fibrin hydrogel, that is a 3D fibrin hydrogel, which is used as a carrier for coating the hyaluronic acid and the stem cell.

In another embodiment, the statin compound is simvastatin. In a preferred embodiment, the simvastatin is in the dosage form of a microsphere.

In one embodiment, the fibrin hydrogel which contains hyaluronic acid and stem cells, and the simvastatin-containing microsphere may be in separate dosage forms, but administered together when used. Alternatively, the simvastatin-containing microsphere may be administered in a single dosage form after being dissolved in the fibrin hydrogel.

When compared with other commonly used technologies, the use of the pharmaceutical composition provided by the present invention in preparation of a drug for promoting chondrogenesis further has the following advantages:

(1) When hyaluronic acid is used in combination with a statin compound, factors associated with chondrogenesis or factors inducing chondrification of cells, such as bone morphogenetic protein (BMP), cartilage extracellular matrix (such as sGAG or Col II), chondrogenic genes (such as Sox-9, aggrecan or type II collagen), are significantly and effectively potentiated, and thus can be used for the treatment of various diseases associated with cartilage defects/wear.

(2) Currently, many commercially available medications or reagents for treating cartilage defects or for stimulating cartilage growth have the potential of causing osteosclerosis, which is probably caused by over-expression of chondrogenic factors. However, the pharmaceutical composition of the present invention not only significantly increases cartilage formation or induces the expression of factors associated with chondrogenesis in cells, but further inhibits the expression of osteosclerosis factors, for example, reduces the expression level of osteocalcin, to prevent the occurrence of osteosclerosis.

### EXAMPLES

The present invention may be embodied in a variety of contents and is not limited to the examples below. The examples below are merely representative of various aspects and features of the present invention.

### Example 1

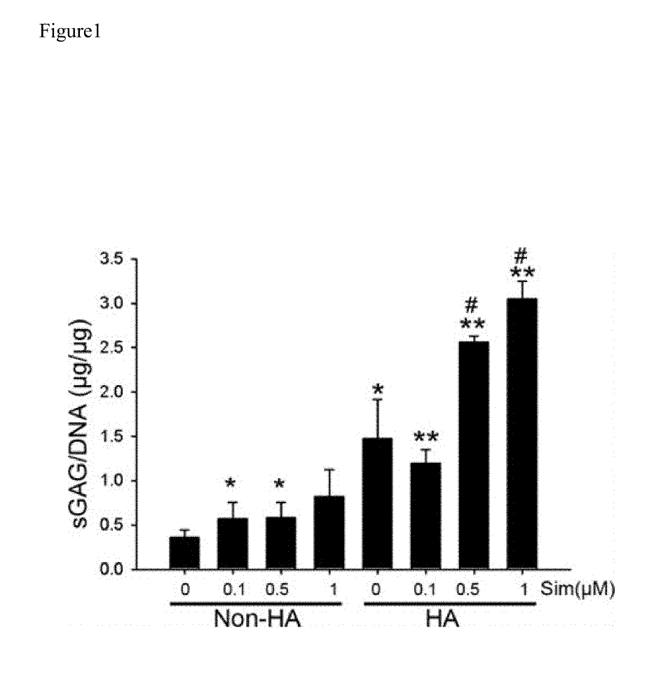

Sulfated glycosaminoglycan (sGAG) is a major extracellular matrix of articular cartilage. In order to test whether simvastatin can promote the expression of sGAG in human adipose-derived stem cells (hADSCs) and its optimal concentration, the present invention first prepared hyaluronic acid (HA) coated wells by coating hyaluronic acid dissolved in 1 ml of phosphate buffer (PBS, 1% w/w) on a 24-well plate, standing at 37° C. for 48 hours, then washed with PBS two times. Human adipose-derived stem cells were seeded in wells containing 500 μl of basal medium at a density of 1×105 cells per well, wherein the basal medium contains Dulbecco's modified Eagle's medium (DMEM), and was additionally supplemented with 5% fetal bovine serum (FBS), 1% nonessential amino acids and 100 U/ml of penicillin/streptomycin (Gibco-BRL, Grand Island, N.Y.). During the culturing period, simvastatin at different concentrations (0.1 to 1 μM) was added to the well plate, and the medium was replaced with fresh one every two days. On day 7 after the cultivation started, cells were collected for dimethylmethylene blue (DMMB) analysis with Blyscan sulfated glycosaminoglycan reagent (Biocolor, Antrim, UK), and for standardization of sulfated glycosaminoglycan (sGAG) formation with DNA. The results were shown in FIG. 1.

The present invention tested the effect of simvastatin on the formation of sulfated glycosaminoglycans (sGAG) in human adipose-derived stem cells in the presence or absence of hyaluronic acid. As show in FIG. 1, in the absence of hyaluronic acid (Non-HA), the expression levels of sGAG in human adipose-derived stem cells were all enhanced when treated with 0.1-1 μM of simvastatin (0 μM: 0.3 μg/μg; 0.1 μM: 0.5 μg/μg; 0.5 μM: 0.5 μg/μg; 1 μM: 0.7 μg/μg); in the presence of 1% hyaluronic acid, the expression levels of sGAG were further enhanced when treated with 1 μM of simvastatin (0 μM: 1.4 μg/μg; 0.1 μM: 1.3 μg/μg; 0.5 μM: 2.5 μg/μg; 1 μM: 2.8 μg/μg). This experiment showed that when 1% hyaluronic acid and 1 μM of simvastatin worked together, the expression of sGAG in stem cells was significantly and preferably enhanced to induce chondrification of cells, thereby promoting cartilage formation.

### Example 2

Human adipose-derived stem cells (hADSCs) were cultured in wells coated with hyaluronic acid (HA) and without hyaluronic acid (non-HA), and treated with or without 1 μM of simvastatin (SIM) for 1 to 5 days. The present invention was therefore divided into four experimental groups: (1) No hyaluronic acid (Non-HA) group: human adipose-derived stem cells were cultured in wells not coated with hyaluronic acid and not treated with simvastatin (1 μM); (2) No hyaluronic acid plus simvastatin (Non-HA+Sim) group: human adipose-derived stem cells were cultured in wells not coated with hyaluronic acid but treated with simvastatin (1 μM); (3) Hyaluronic acid (HA) group: human adipose-derived stem cells were cultured in wells coated with hyaluronic acid but not treated with simvastatin (1 μM); and (4) Hyaluronic acid plus simvastatin (HA+Sim) group: human adipose-derived stem cells were cultured in wells coated with hyaluronic acid and treated with simvastatin (1 μM).

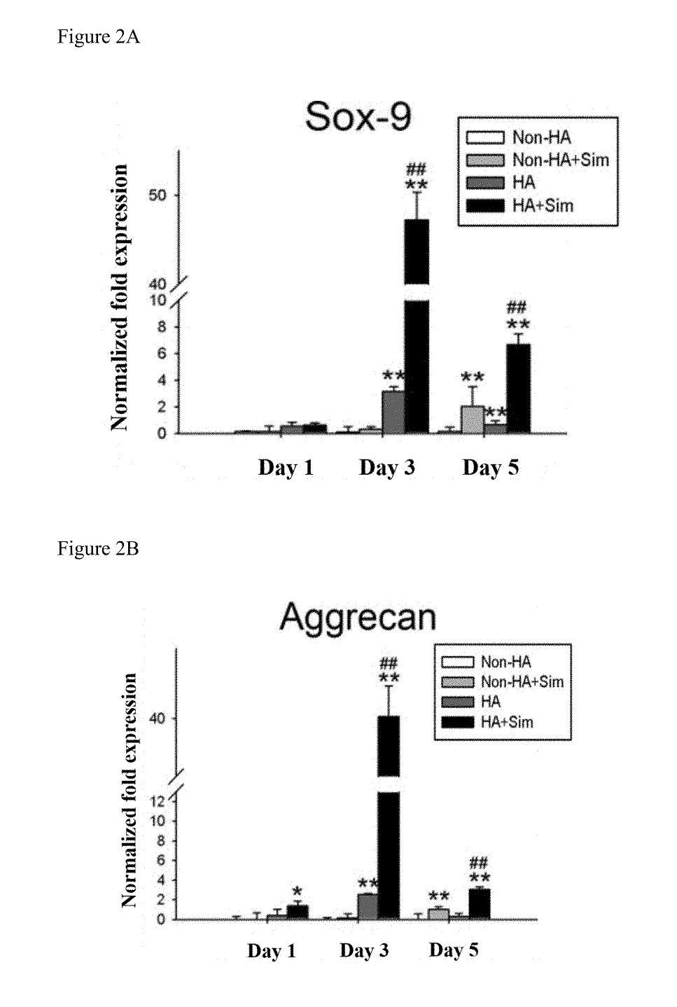

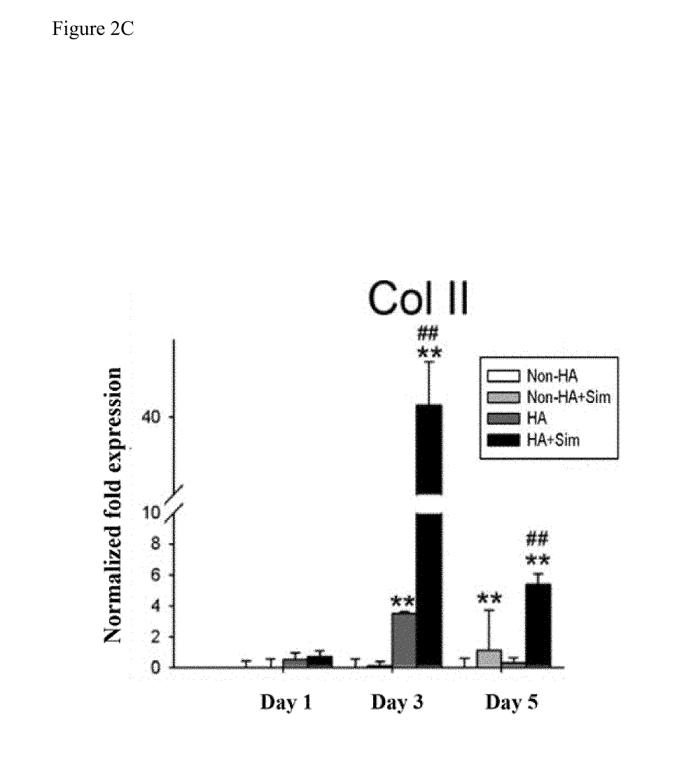

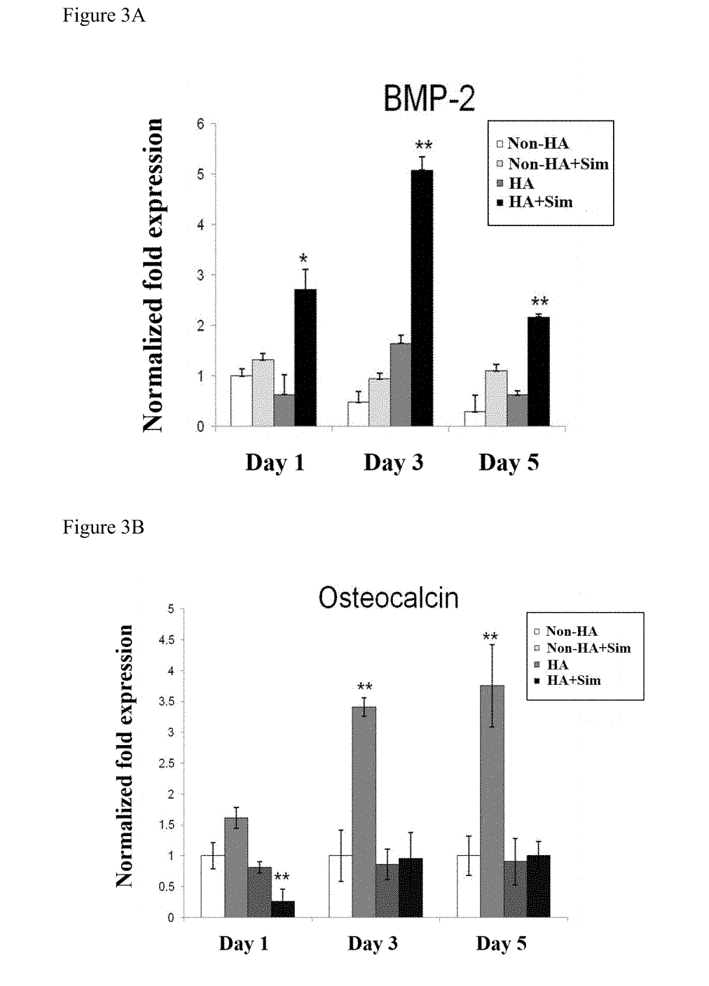

Cells were harvested on designated days and analyzed for the expression of BMP-2 and chondrogenic genes using real-time PCR. Total RNA of these cells was extracted by following the manufacturer's instructions and using TRIzol reagent (Gibco BRL, Rockville, Md.). In short, a reaction volume of 20 μl was taken which contained 0.5 to 1 μg of total RNA, and then RNA was reverse-transcribed into cDNA by using the SuperScript First-Strand Synthesis System (Invitrogen). Real-time PCR reactions were performed and monitored by using iQTM SYBR Green® supermix (Bio-Rad Laboratories Inc, Hercules, Calif.) and a quantitative real-time PCR detection system (Bio-Rad Laboratories Inc, Hercules, Calif.). The cDNA sample (2 μl was taken to be used in a total volume of 25 μl for each reaction) was used for analysis of those genes of interest and the reference gene glyceraldehyde 3-phosphate dehydrogenase (GAPDH) was used as a control group. The expression level of each of the target genes, as previously described, was calculated as 2-ΔΔCt. For each gene of interest, four readings were performed for each of the experimental samples, each experiment was repeated for at least three times. Applied primer sets were as follows: (1) bone morphogenetic protein-2 (BMP-2), forward: CGAATGACTGGATTGTGGCT, reverse: TGAGTTCTGTCGGGACACAG; (2) Sox-9, forward: CTT CCG CGA CGT GGA CAT, reverse: GTT GGG CGG CAG GTA CTG; (3) Collagen type II (Col II), forward: CAA CAC TGC CAA CGT CCA GAT, reverse: TCT TGC AGT GGT AGG TGA TGT TCT; (4) aggrecan, forward: ACA CGT GGG GAC AGT ATT GG, reverse: GTG GAA AGA TGC GGT GGT TT; (5) GAPDH, forward: TCT CCT CTG CAT TCA ACA GCGAC, reverse: CCC TGT TGT TGC AGC CAA ATT C; and (6) osteocalcin, forward: GTG CAG AGT CCA GCA AAG GT, reverse: CGA TAG GCC TCC TGA AAG C, the results are shown in FIG. 2 and FIG. 3.

The present invention first tested whether the composition of 1% of hyaluronic acid (HA) and 1 μM of simvastatin stimulated the expression of chondrogenic genes (such as Sox-9, aggrecan and Col II) and their preferred concentrations. As shown in FIGS. 2A to 2C, in the absence of hyaluronic acid (Non-HA), 1 μM of simvastatin failed to effectively enhance the expression of chondrogenic genes (Non-HA+Sim); however, in the presence of hyaluronic acid, better expression levels of chondrogenic genes were achieved when 1 μM of simvastatin (HA+Sim) was administered, that is, increased expression levels of Sox-9, aggrecan and Col II. This experiment showed that when 1% of hyaluronic acid and 1 μM of simvastatin worked together, the expression of chondrogenic genes in stem cells was significantly and preferably enhanced.

The present invention tested whether the composition of 1% of hyaluronic acid and 1 μM of simvastatin stimulated the expression of BMP-2 and osteocalcin in the presence or in the absence of hyaluronic acid. As shown in FIG. 3A, when the expression level of BMP-2 on the first day of the non-hyaluronic acid group (Non-HA) was regarded as 1 for the purpose of comparison, in the absence of hyaluronic acid (Non-HA) the expression of BMP-2 gene was not significantly enhanced by giving 1 μM of simvastatin (day 1: Non-HA group: 1 fold, Non-HA+Sim group: 1.2 fold; day 3: Non-HA group: 0.5 fold, Non-HA+Sim group: 0.8 fold; day 5: Non-HA group: 0.2 fold, Non-HA+Sim group: 1.1 fold.) In the presence of 1% of hyaluronic acid and 1 μM of simvastatin was given (HA+Sim), the expression of BMP-2 was significantly increased (day 1: HA: 0.5 fold, HA+Sim: 2.8 fold; day 3: HA: 1.9 fold, HA+Sim: 5 fold; day 5: HA: 0.4 fold, Non-HA+Sim: 2.1 fold), this experiment showed that when 1% of hyaluronic acid and 1 μM of simvastatin worked together, the expression of BMP-2 gene was significantly increased.

In terms of gene expression level of osteocalcin, in FIG. 3B, when the expression level of osteocalcin on the first day of the Non-HA group was regarded as 1 for the purposes of comparison, 1 μM of simvastatin was given in the absence of hyaluronic acid (Non-HA), the expression of osteocalcin gene was significantly increased from day 1 to day 5, however, when 1 μM of simvastatin was given in the presence of HA (HA+Sim), the expression of osteocalcin induced by simvastatin was effectively reduced from day 1 to day 5. The above results indicated that when hyaluronic acid and simvastatin worked together the expression of osteocalcin was reduced, thereby decreasing the occurrence of osteosclerosis.

### Example 3

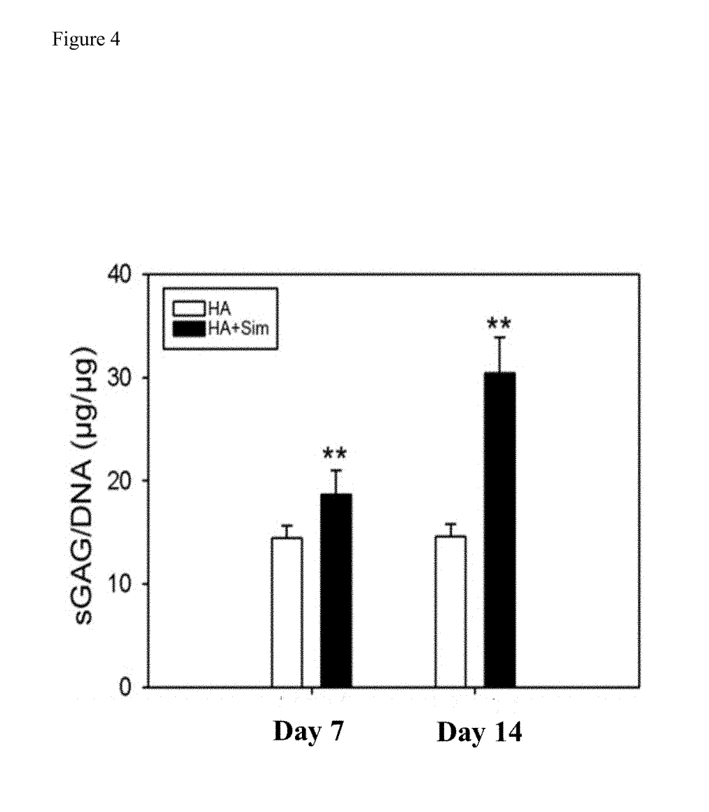

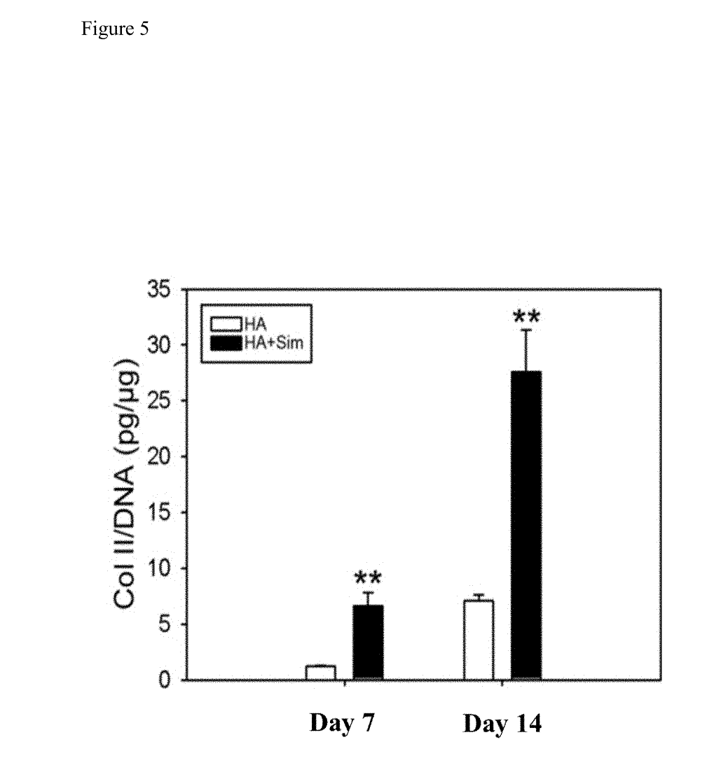

30 μL of human adipose-derived stem cells (5×106 cells) suspended in 1% of hyaluronic acid was mixed with 120 μl of fibrin solution (100 mg/ml), and then placed in a teflon mold (5.5 mm in depth and 5.5 mm in diameter). 40 μl of bovine thrombin (300 U/ml) dissolved in 40 mM of CaCl2 was added into the mold and mixed well with the cell/fibrin solution. The mixture was incubated for 15 minutes at room temperature until a hydrogel was formed. After the hydrogel was formed, the three-dimensional fibrin hydrogel carrier containing human adipose-derived stem cells and hyaluronic acid (hyaluronic acid/fibrin hydrogel) was removed from the Teflon mold, and then moved to a 24-well plate, cultivated with 1 ml of basal medium (DMEM), wherein the basal medium contained Dulbecco's modified Eagle's medium (DMEM), and was additionally supplemented with 5% of fetal bovine serum (FBS), 1% of nonessential amino acids and 100 U/ml of penicillin/streptomycin (Gibco-BRL, Grand Island, N.Y.). The human adipose-derived stem cells (hADSCs) were cultured in the hyaluronic acid/fibrin hydrogel and treated with or without 1 μM of simvastatin for 7 or 14 days. Therefore, there were two experimental groups in the present invention: (1) Hyaluronic acid (HA) group: human adipose-derived stem cells were cultured in the hyaluronic acid/fibrin hydrogel but not treated with simvastatin (1 μM); and (2) Hyaluronic acid plus simvastatin (HA+Sim) group: human adipose-derived stem cells were cultured in the hyaluronic acid/fibrin hydrogel and treated with simvastatin (1 μM). On days 7 and 14, hydrogels were collected and incubated in papain solution (300 μg/ml) for 18 hours at 60° C. Then, 50 μl of the cell extract was obtained from each experimental group to detect the synthesis of sGAG by using the DMMB assay or to analyze type II collagen by using Type II Collagen Detection Kit (Chondrex Inc. WA, USA). The results were shown in FIGS. 4 and 5.

When coated with the three-dimensional hyaluronic acid hydrogel, whether 1 μM of simvastatin would effectively enhance the formation of two major extracellular matrix in mesenchymal stem cells, namely, sulfated glycosaminoglycans (sGAG) and type II collagen (Col II). As for the expression level of sGAG, on day 7, the hyaluronic acid (HA) group was 14 μg/μg, the hyaluronic acid plus simvastatin (HA+Sim) group was 18 μg/μg; on day 14, the hyaluronic acid group was 14 μg/μg, and the hyaluronic acid plus simvastatin group was 30 μg/μg (as shown in FIG. 4). As for the expression level of Col II, on day 7, the hyaluronic acid (HA) group was 2 pg/μg, the hyaluronic plus simvastatin (HA+SIM) group was 6 pg/μg; on day 14, the hyaluronic acid group was 6.5 μg/μg and the hyaluronic acid plus simvastatin group was 27 μg/μg (as shown in FIG. 5). The above results showed that when coated with the three-dimensional hyaluronic acid/fibrin hydrogel, 1 μM of simvastatin (HA+Sim) effectively enhanced the expression of two major extracellular matrix (sGAGs and Col II) in stem cells.

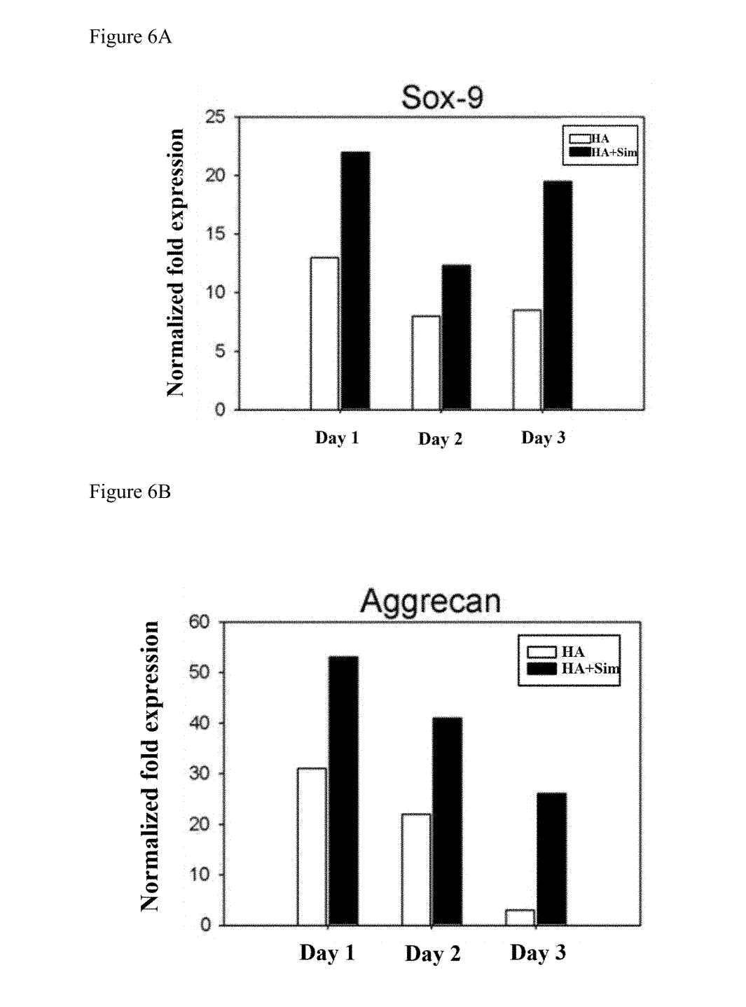

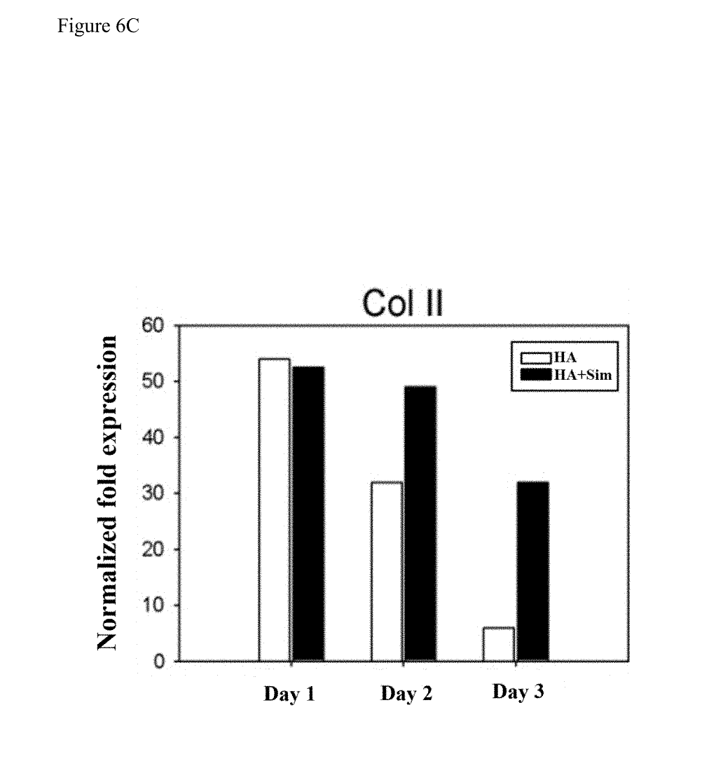

In addition, the present invention further utilized hyaluronic acid/fibrin hydrogel to culture human adipose-derived stem cells and treated them with or without 1 μM of simvastatin for 1 to 5 days to analyze the expression of chondrogenic genes (Sox-9, Aggrecan and type II collagen (Col II)). Therefore, in the present invention, there were two experimental groups: (1) Hyaluronic acid (HA) group: human adipose-derived stem cells were cultured in the hyaluronic acid/fibrin hydrogel but not treated with simvastatin (1 μM); and (2) hyaluronic acid plus simvastatin (HA+Sim) group: human adipose-derived stem cells were cultured in the hyaluronic acid/fibrin hydrogel and treated with simvastatin (1 μM). The cells were collected on designated days and the expression of chondrogenic genes was analyzed by using real-time PCR. The results are shown in FIG. 6.

When coated with the three-dimensional hyaluronic acid hydrogel, whether 1 μM of simvastatin would effectively and preferably enhance the expression of chondrogenic genes in mesenchymal stem cells. As shown in FIGS. 6A to 6C, when stems cells were coated with the 3-dimensional hyaluronic acid/fibrin hydrogel, the effect of better expression of the chondrogenic genes was obtained when 1 μM of simvastatin (HA+Sim) was administered, that is, the expression of Sox-9, aggrecan and Col II was increased. This experiment showed that when coated with the three-dimensional hyaluronic acid hydrogel, the effect of better expression of the chondrogenic genes in stem cells could be obtained when 1 μM of simvastatin (HA+Sim) was administered.

### Example 4

40 mg of simvastatin (Merck & Company, Rahway, N.J., USA) was dissolved in 1 ml of ethanol and then mixed with 1.5 ml of 0.1 N NaOH. The simvastatin stock solution was heated to 50° C. for 2 hours and then the pH value was neutralized with 1 N of hydrochloric acid (HCl, pH=7.4, Sigma-Aldrich, St Louis, Mo., USA) to form a 10 mM of stock solution. Microspheres were then prepared by a water-in-oil-in-water (w/o/w) double emulsion technique. In short, 16 mg of hydroxyapatite (HAp) powder was dissolved in phosphate buffered saline (PBS) solution to form a first water-phase solution. 250 μl of simvastatin stock solution, 50 mg of surfactant Span 80 and polylactic-co-glycolic acid (PLGA) 50/50 (P2191, molecular weight: 30,000-60,000, Sigma-Aldrich) were mixed in dichloromethane solution to form an oil phase. The first water-phase solution and the oil phase solution were mixed with each other and stirred at 1000 rpm for 15 minutes to form a first water-in-oil (w/o) emulsion. The first water-in-oil (w/o) emulsion was added into 20 ml of the second water-phase solution and mixed with a 1% (w/v) of dichloromethane solution to form a second double emulsion (w/o/w). The second double emulsified (w/o/w) suspension was stirred at room temperature for 30 minutes to harden the microspheres. While stirring the suspension, exhaustion was essential to evaporate dichloromethane and to harden the microspheres. Finally, the microspheres were collected by centrifugation and washed three times with 0.1% of polyvinyl alcohol before being lyophilized in a lyophilizer.

All experimental animals were provided by Taitung Animal Propagation Station (TAPS). Animals were anesthetized by 0.15 mg/kg of Ketamine and sedative xylazine administered via intramuscular injection prior to surgery. During surgery, animals were given 5% of inhalational Isoflurane to maintain anesthesia. Therefore, after the miniature pigs had been anesthetized the operation was done on left legs. Full-thickness cartilage defects (6 mm in diameter) were created on the medial condyles of the left leg by using a trephine but which did not penetrate through the subchondral bone. After surgery, the hyaluronic acid/fibrin hydrogel containing 1 mg of simvastatin microspheres was implanted into the cartilage defect site which was covered with a periosteum obtained from the tibia for suturing and repairing the structure. Finally, the surgical wound site was stitched together layer by layer. After surgery, painkiller Ultracet (Tramadol 37.5 mg/Acetaminophen 325 mg) was given orally to the animals once daily, two tablets each time for 5 days. At week 12, the effect of treatment was monitored by observation.

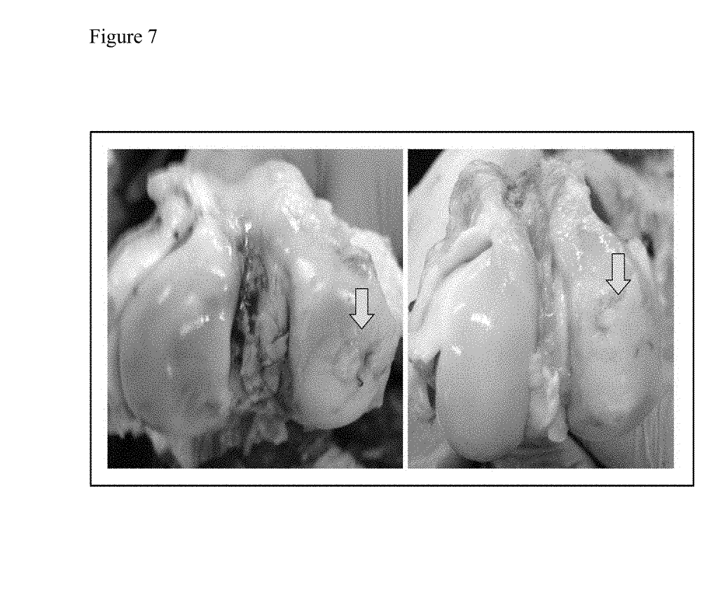

As shown in FIG. 7, when the three-dimensional hyaluronic acid hydrogel, which contained mesenchymal stem cells and simvastatin-containing microspheres, was administered to the articular cartilage defect site of the miniature pigs, it was found that after 12 weeks the articular cartilage defect site was significantly improved. The above results indicated that the mesenchymal stem cells coated with three-dimensional hyaluronic acid hydrogel and the simvastatin-containing microspheres had the effect of repairing and improving the articular cartilage defects.

The detailed description is the detailed description of one preferred embodiment of the present invention, which is not intended as limitations of the present invention. Therefore, It will be readily apparent to a person skilled in the art that varying substitutions and modifications may be made to the invention disclosed herein without departing from the scope and spirit of the invention.

What is claimed is:

1. A use of a pharmaceutical composition in the preparation of a drug for promoting chondrogenesis, wherein the pharmaceutical composition comprises a hyaluronic acid mixture and a statin compound.

2. The use of claim 1, wherein the hyaluronic acid mixture comprises hyaluronic acid, fibrin and a stem cell.

3. The use of claim 2, wherein the fibrin forms a hydrogel.

4. The use of claim 2, wherein the stem cell is human adipose-derived stem cell.

5. The use of claim 1, wherein the statin compound is simvastatin.

6. The use of claim 1, wherein the simvastatin is in the dosage form of a microsphere.

7. The use of claim 1, wherein the pharmaceutical composition further inhibits osteosclerosis of the generated chondrocytes.

8. The use of claim 7, wherein the osteosclerosis of the generated chondrocytes is caused by inhibiting the expression of osteocalcin.

9. The use of claim 1, wherein chondrogenesis is promoted by increasing the expression of bone morphogenetic protein-2 (BMP-2).

10. The use of claim 1, wherein chondrogenesis is promoted by increasing the expression of sulfated glycosaminoglycans (sGAG).

11. The use of claim 1, wherein chondrogenesis is promoted by increasing the expression of chondrogenic genes comprising Sox-9, aggrecan or type II collagen.

12. The use of claim 2, wherein the concentration of the hyaluronic acid in the pharmaceutical composition is 1 wt %.

13. The use of claim 5, wherein the concentration of simvastatin in the pharmaceutical composition is 1 μM.
